(19) United States
(12) Patent Application Publication
(10) Pub. No.: US 2021/0322586 A1
(43) Pub. Date: Oct. 21, 2021

(54) Pocket sterilizer

(71) Applicant: Jerry Forrester, Toronto (CA)

(72) Inventor: Jerry Forrester, Toronto (CA)

(21) Appl. No.: 16/873,461

(22) Filed: Apr. 17, 2020

(51) Int. Cl. A61L 2/00; A61L 2/20

(52) U.S. Cl. CPC A61L 2/0094; A61L 2202/16; A61L 2202/15; A61L 2/20

(57) ABSTRACT

A pocket for sterilizing in which hands and objects can be sterilize comprising,
A hand area 12 for accommodating hands and objects.
A plurality of canisters 40 and 50 containing gas and air for cleaning.
A backpack 14 having straps 14A and pockets for holding the canisters.
A jacket 15 which houses a skirt 17 which houses a cylindrical member 80, a membrane 12 and a pump 20, and
A vest 18 for housing components of the invention configured for, use as a portable and wearable apparatus to clean hands and objects.

### CROSS-REFERENCE TO RELATED APPLICATION

Not Applicable.

### STATEMENT REGARDING FEDERALLY SPONSORED RESEARCH OR DEVELOPMENT

Not Applicable.

### REFERENCE TO SEQUENCE LISTING, A TABLE, OR A COMPUTER PROGRAM LISTING COMPACT DISC APPENDIX

Not Applicable.

### BACKGROUND

### Field of the Invention

The present invention relates to a pocket or pockets for sterilizing human hands and objects.

### Description of Prior Art

Inventors and manufacturers of machines for sterilizing provided stationary machines and means for sterilizing. Companies also provide many types of solutions and other means to be used for sterilizing hands and instruments.

### DETAILED DESCRIPTION OF THE INVENTION

This invention relates to a method for sterilizing human hands and objects that may need to be sterilized comprising:

A plurality of cylinders for providing compress sterilizing solution and sterilized air and or moisturizing air.

A dispenser(s) chamber(s) in the form of a tube-like vessel for dispensing the predetermined amount of compress sterilizing gas and sterilized air comprising,

A plurality of attachments such as,

A plurality of hoses, fittings and nozzles of predetermined sizes in order to dispense the gas or gases for sterilizing and sanitizing the hands and objects which need to be cleaned.

an outer cylindrical member composed of a rigid plastic and can be composed of a resilient material such as plastic or a fabric suitable for guiding and keeping in predetermined positions stationary and moveable members of the embodiment and a hand area for accommodating hands and objects.

The invention further comprising, a plurality of triggers and linkages, and their functions includes,
a. Allowing a locking mechanism of the outer cylinder to be locked position,
b. Triggering the opening of a valve for sterilizing,
c. Triggering the closing of the valve for sterilizing,
d. Triggering the opening of a valve for sterilized air flow,
e. Triggering the closing of the valve for sterilized air flow, and
f. Triggering the unlocking of the locking mechanism of the outer cylinder,
g. A plurality of the valves attached thereon and connects with the cylinders for opening and closing gas and air flow.

The invention further comprising, a pump or inner telescopic member for, guiding moving and keeping in predetermined positions, attached moveable members, such as

A plurality of gates for triggering the turning on and off a mechanism for locking and the air and sterilizing gas flow,

A windbag having, a predetermined amount of sealed air and is, configured for accommodating air forced into it by the pump,

A plurality of Valves for controlling pumped sealed air flow, which includes

a valve of the telescopic pump which allow for circulation of pressurized air of the pump and causes rotation of,

An exhaust fan that pushes out the used sterilizing gas and sterilized aid,

A pocket(s) which houses the hand area, the membrane and the outer cylinder which houses the pump, and connects with a plurality of valves, triggers, linkages and the dispenser by way of a plurality of hoses and fittings, and is for inserting what must be sterilized,

A Jacket(s) which houses the pocket and the cylinders and can be worn with harnesses and or straps. The jacket can include a backpack for holding the cylinders containing air and gas; it can also be configured for a parent to carry in a bag to clean children hands. The jacket also houses a skirt which seals the hand area and outer cylinder therein, and it is between the outer cylinder and the jacket.

### SUMMARY

In accordance with the present invention, a wearable sterilizer composed of non-conductive materials like plastics and rubber and can be composed of some conductive materials, to be use by humans for sterilizing their hands and other objects.

This invention employs compress sterilizing gases for sterilizing and compress dry sterilized air and or compress moisturizing gas in order to clean hands and objects.

The invention further comprises of, a plurality of, cylinders, gates, triggers, valves, fittings, hoses, a pump or telescopic member, a skirt, a jacket and a vest.

The pocket sterilizer uses outside force, like a hand being inserted into its pocket and pushes down, which forces air through a stationary gate having a one-way valve for allowing air into the windbag. The air stored in the windbag is releasable by way of another one-way valve which, allows the air to reenter into the telescopic pump returning it to its original position. Downward movements of the pump by a person cause, certain triggers to cause certain valves to open or close and to cause compress air to react by pushing back on the inserting force thereby, resetting or allowing invention to return to a ready to start position.

### Objects and Advantages

Accordingly, besides the objects and advantages of the pocket sterilizer described in this application for patent, several objects and advantages of the present invention are:
a. To provide a wearable sterilizer that can be use by doctors, dentists, nurses, helpers in the medical fields and the public,
b. To provide a wearable pocket sterilizer that can be produce quickly and in large numbers.
c. To provide a pocket sterilizer that can be affordable for the general public.
d. To provide a pocket sterilizer that can be produced from durable and resilient materials light enough to be carried around while moving around.
e. To provide a pocket sterilizer that is easy to wear and use at the same time.

A further object and advantage of the pocket sterilizer is,
f. To provide an apparatus that a person can wear over their close as a sterilizer that will, provide a means for sterilizing that is convenient, affordable and available to everyone.

### DRAWING FIGURES

In the drawings which illustrate embodiments of the invention,

FIG. 1 is a perspective view of preferred embodiments of the present invention having a transparent outer cylinder and in an initial stage.

FIG. 1A, is a perspective view of the preferred embodiment illustrating the pump in a second stage and the positions of its components.

FIG. 1B, is a perspective view of the preferred embodiment showing the pump in a third stage, showing the positions of its components compare to FIGS. 1 and 1A drawings.

FIG. 1C, is a perspective view of the preferred embodiment showing the pump in a fourth stage, and the new positions of its components compared to FIGS. 1, 1A and 1B.

FIG. 1D, is a perspective view of the embodiment showing the pump in a fifth stage, and the new positions of its components.

FIG. 1E, is a perspective view of the embodiment showing the pump in a sixth initial stage, and the positions of its components.

FIG. 2, is a perspective view of the embodiment showing a pair of the embodiment.

FIG. 3. is a perspective view of the embodiment showing a transparent outer cylinder and the various components of the invention.

FIG. 4, is a perspective view of an additional embodiment of the invention having a coil spring.

FIG. 5, is a perspective view of the embodiment and a flowchart illustrating how the invention works.

FIG. 6 is a perspective view of a section of the invention.

FIG. 7 is a perspective view of a part of an additional embodiment of the invention.

FIG. 8 is a perspective view of two cinctures of the invention.

FIG. 9 is a perspective view of a membrane of the invention.

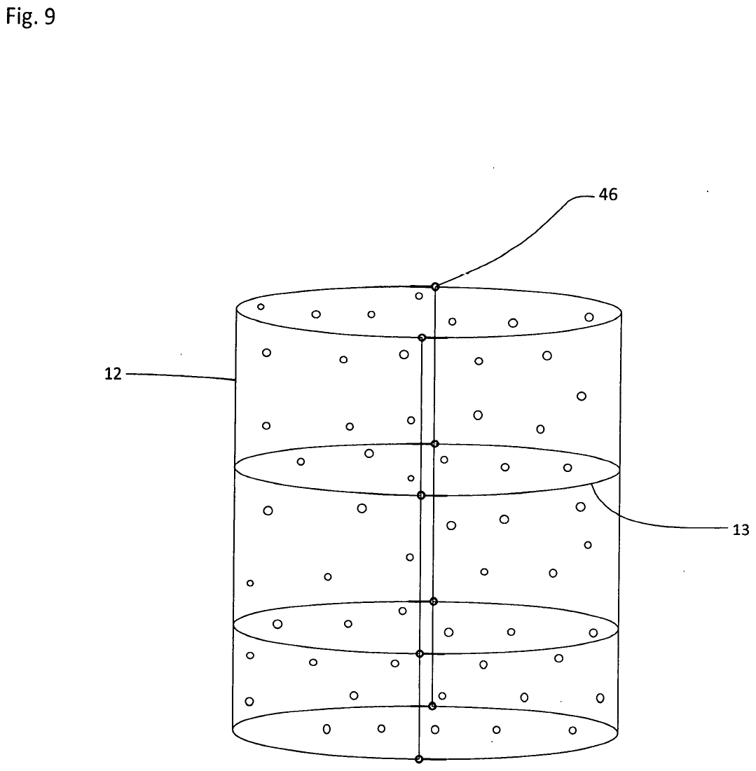

Reference Numerals:

| | |
|---|---|
| Embodiment | 10 |
| Elastic ring(closer). | 11 |
| Membrane, (around hand area). | 12 |
| Hand area | 12A |
| Cincture | 13 |
| Backpack | 14 |
| Straps | 14A |
| Jacket | 15 |
| Pocket | 16 |
| Skirt | 17 |
| Vest | 18 |
| Coil spring | 19 |
| Pump | 20 |
| Push-Top, (or top end of the pump). | 21 |
| Base, (or closed ends of the pump cylindrical members). | 21A |
| First telescopic cylindrical member, (of inner and upper | 22 |
| member of pump). | |
| First ring gate, (of pump First upper and outer member). | 22A |
| Second cylindrical member, (or middle member of pump). | 23 |
| Second ring gate, (of pump second or middle member). | 23A |
| Third cylindrical member, (or outermost member of pump). | 24 |
| Stationary Gate, (of the pump bottom). | 24A |
| Windbag, (attached to the bottom of pump). | 25 |
| Exhaust fan, (attached to the skirt and the jacket). | 30 |
| Exhaust air filter | 31 |
| Gear | 32 |
| Housing, (for the fan filter and gear). | 33 |
| Cup hinge | 34 |
| Sterilizing gas cylinder | 40 |
| Larger Hose | 42 |
| Smaller hose | 42A |
| Fittings | 43 |
| First valve (for sterilizing gas flow). | 45A |
| Trigger, (to turn on the sterilizing gas flow). | 45B |
| Trigger, (to turn off the sterilizing gas flow). | 45B |
| Drying/Sanitizing air valve | 45C |
| Trigger, (to turn on drying air and sterilized air valve). | 45B |
| Trigger, (to turn off the sterilized air valve). | 45B |
| One-way valve of the stationary disc or bottom of the pump. | 45E |
| Two-way valve with a bypass of Pump middle- or second- | 45F |
| cylindrical member. | |
| Two-way valve with the bypass of Pump outermost | 45G |
| cylindrical member. | |
| Reducer (one-way valve), at the bottom of pump. | 45H |
| Linkages | 46 |
| Mechanism for locking | 47 |
| Trigger, (to unlock locking mechanism). | 45B |
| Drying/moisturizing gas/air cylinder | 50 |
| Dispenser chamber | 70 |
| Nozzle, (of hose from dispenser). | 72 |
| Outer cylinder. | 80 |
| Holes. | 81 |

### DETAILED DESCRIPTION AND OPERATING STAGES OF THE INVENTION

The pocket sterilizer 10 is of a predetermined form, configured for wearing by humans, so that users of it can sterilize their hands (not showed in the drawings) and or instruments (not showed in the drawings) at regular intervals.

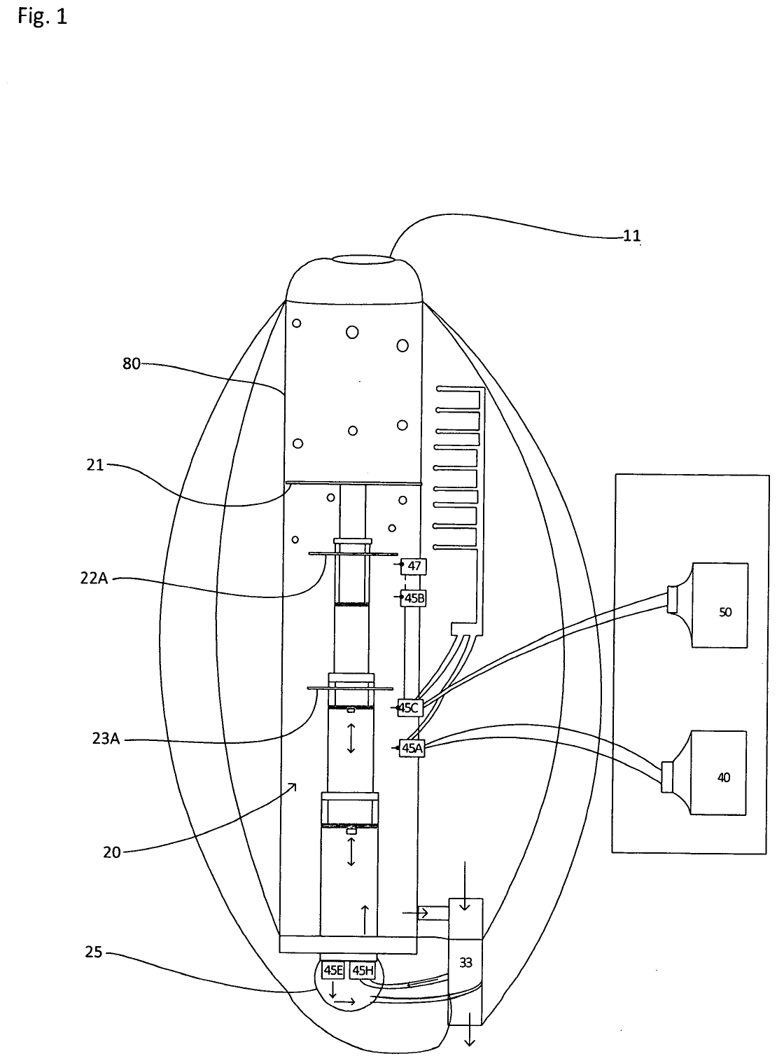

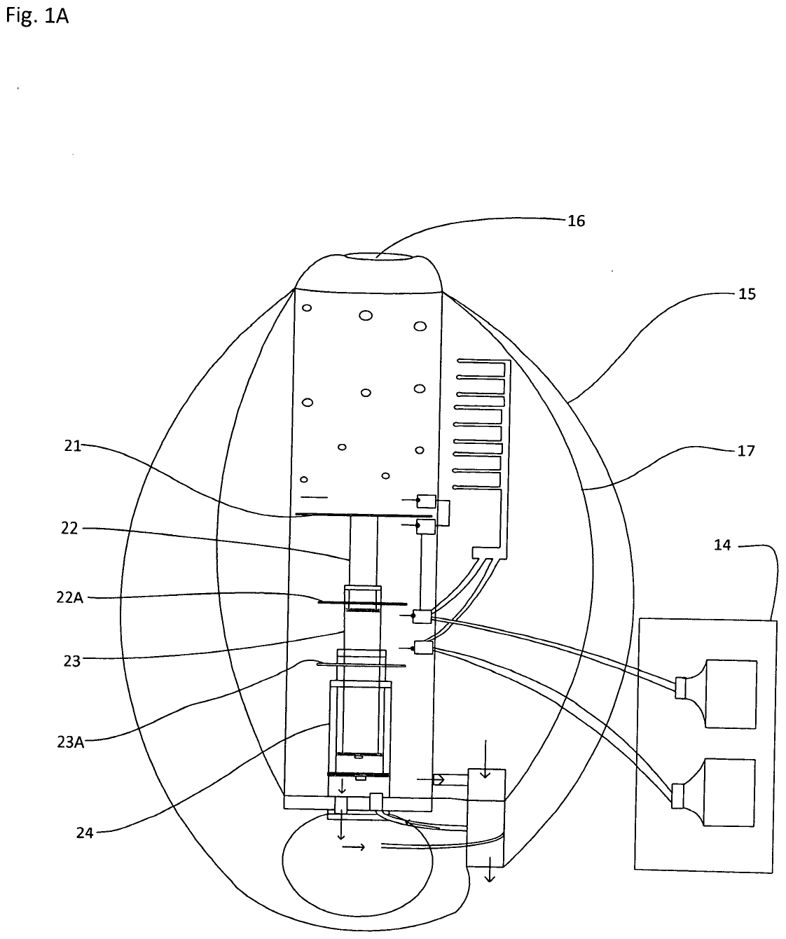

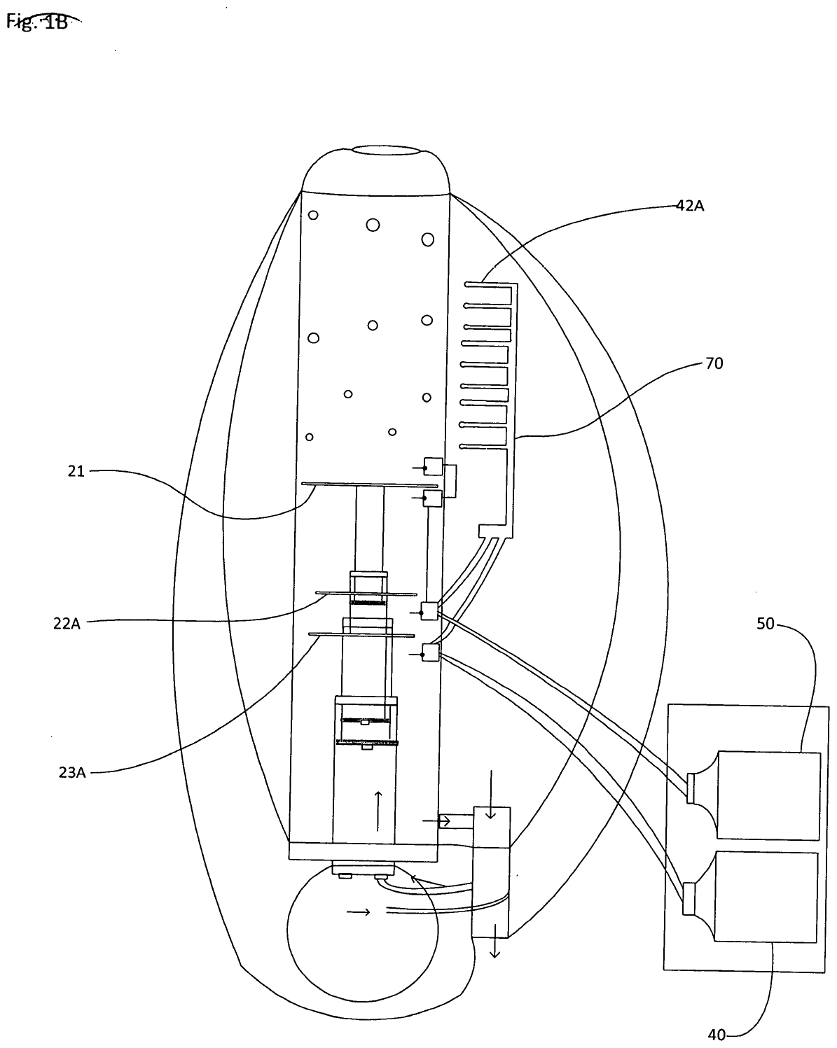

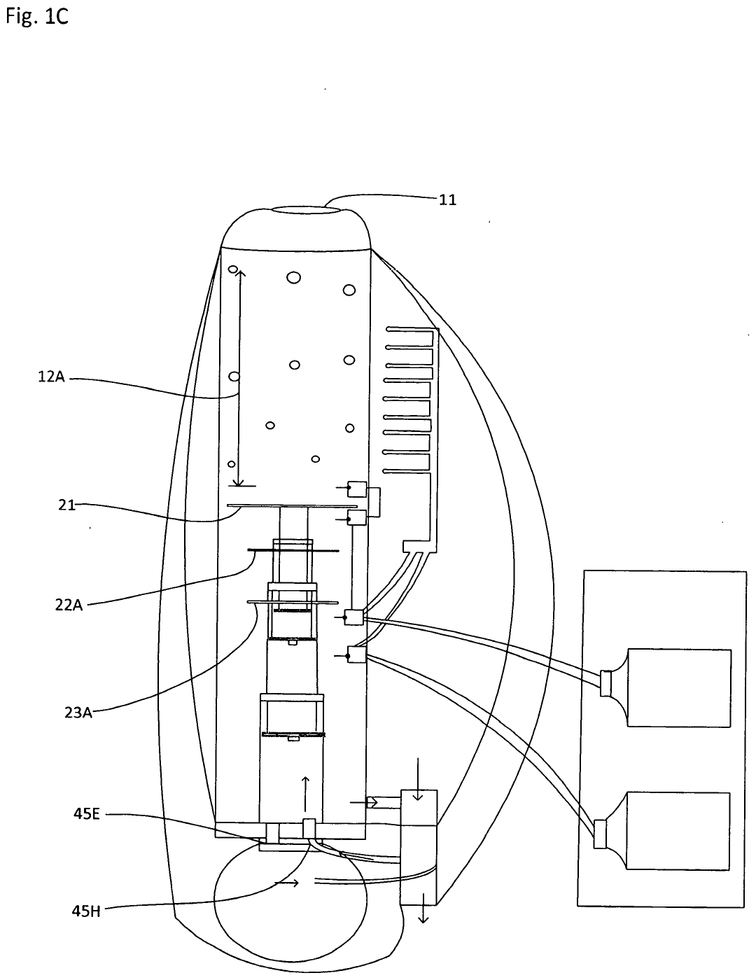

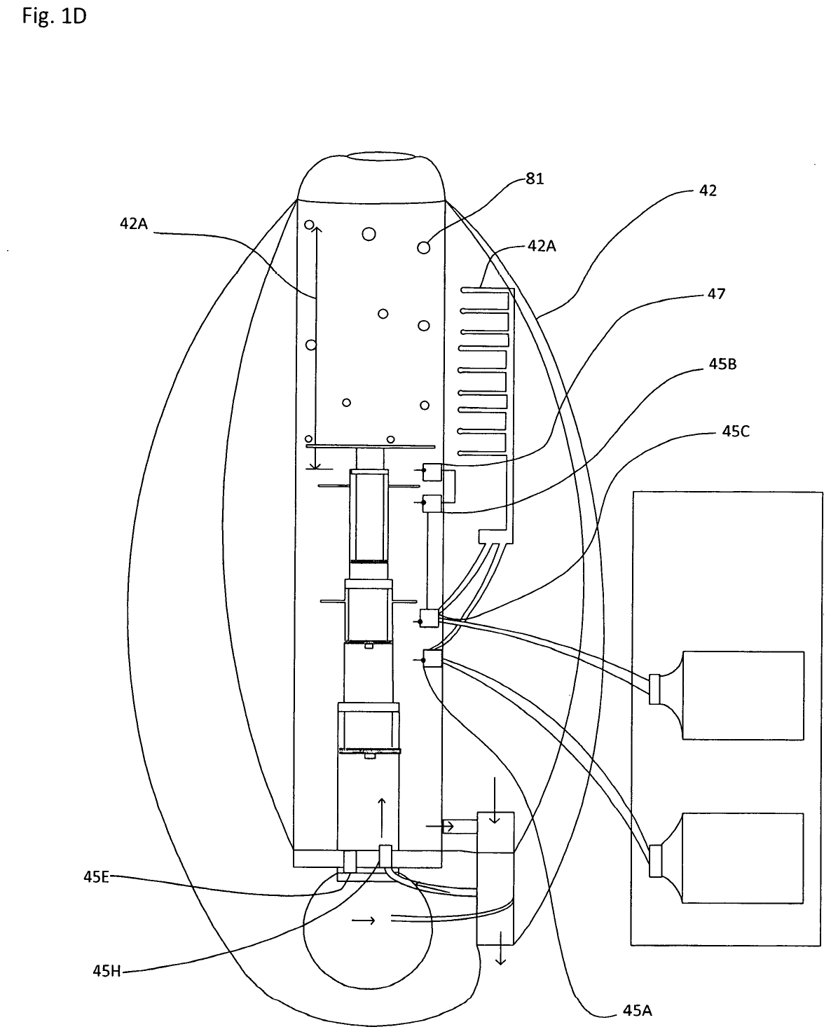

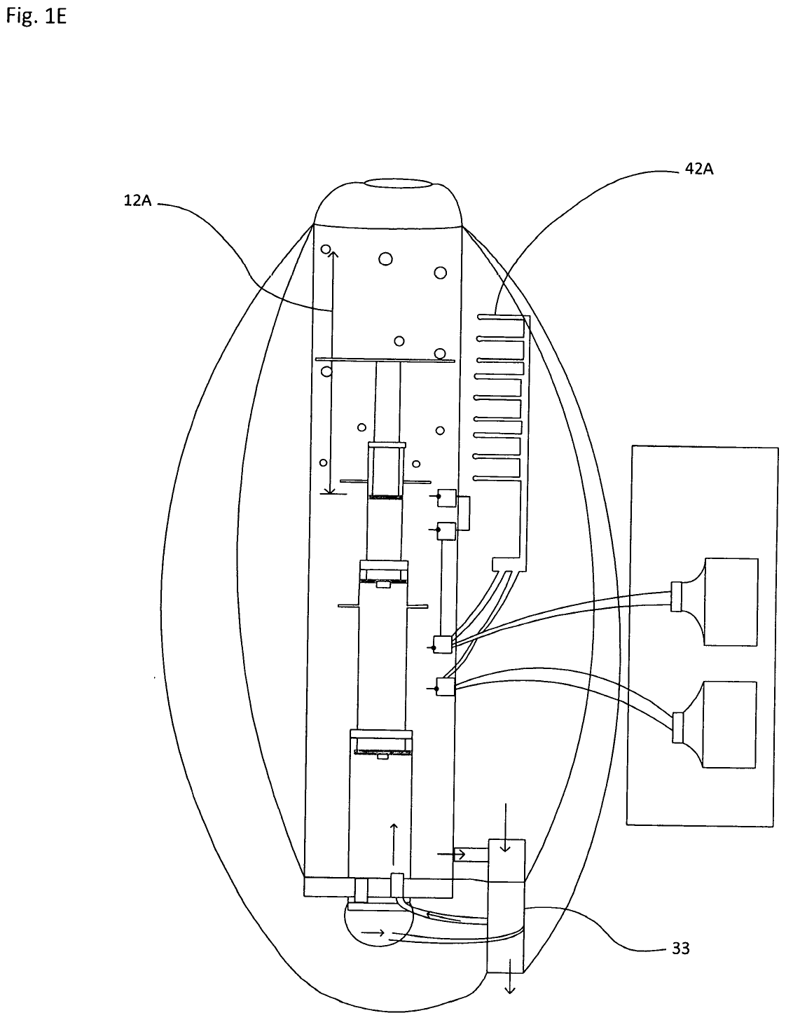

FIG. 1, preferred embodiments of the invention:

The drawing shows the invention in an initial stage with pump 20 in an upward position and further depicted, elastic ring 11 at opening of pocket 16, outer cylinder 80, top 21 of pump, gate 22A of middle member 22 of pump, gate 23A of outer member 23 of pump. The drawing further depicted, valve 45A for turning on and off gas flow and valve 45C for turning on and off cleaning and or moisturizing gas and air flow.

The drawing of FIG. 1A, show the invention at a second stage with pump 20 in a downward locked position with gats 22A and 2A at lower positions relative to their positions at the initial stage. The position of gate 23A in the second stage opens valve 45A for sterilizing.

The drawing of FIG. 1B, show the invention at a third stage with pump 20 still locked while gates 22A and 23A have moved upwardly a bit. The position of gate 23A in the third stage closes valve 45A for sterilizing. The position of gate 22A in the third stage opens valve 45C for cleaning, sanitizing and moisturizing.

The drawing of FIG. 1C, show the invention at a fourth stage with pump 20 still locked while gates 22A and 23A have moved upwardly a bit more. The position of gate 22A in the fourth stage closes valve 45C for cleaning, sanitizing and moisturizing.

The drawing of FIG. 1D, show the invention at a fifth stage with pump 20 in an unlocked stage while gates 22A and 23A have moved upwardly a bit more. The position of gate 22A in the fifth stage unlocked mechanism 47 for locking so as to release top 21 of pump 20. The position of gate 23A in the fifth stage closes valve 45C for cleaning, sanitizing and moisturizing.

The drawing of FIG. 1E, show the invention at a sixth stage with pump 20 in an unlocked and initial upward position.

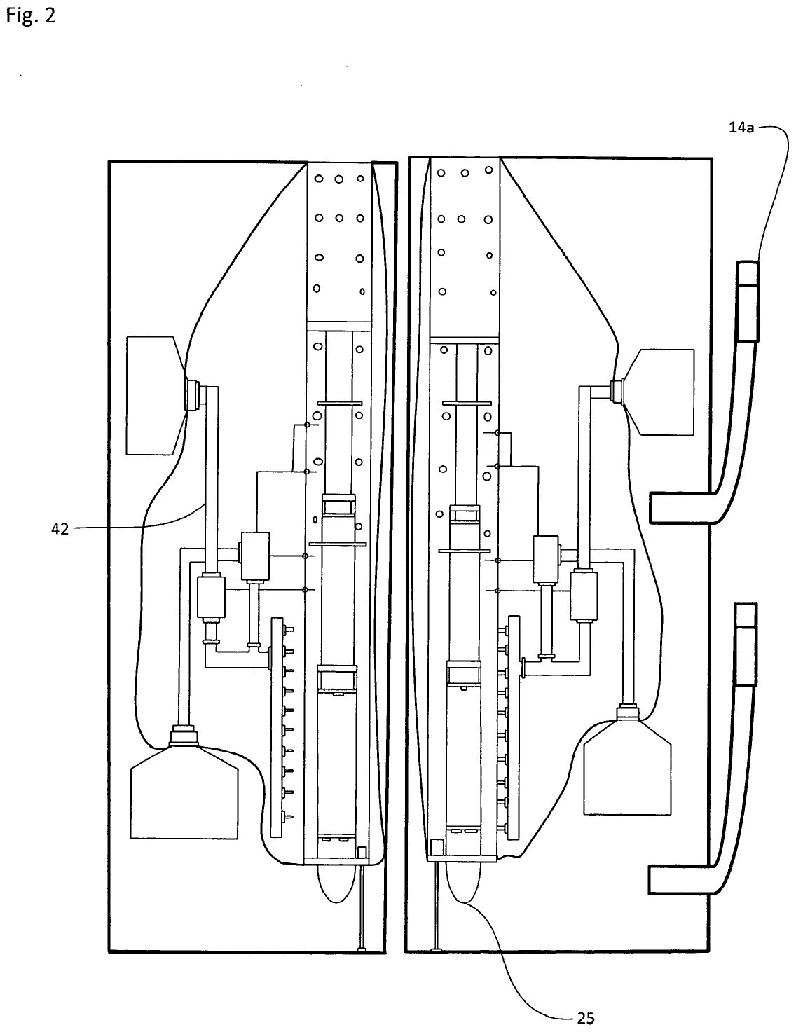

The drawing of FIG. 2, show a pair of the pocket sterilizer 10 comprising, all of the components of the invention. The drawing depicts straps 14A, windbag 25 and hose 42. The drawing further showed, compress sterilizing gas cylinder 40 and compress drying air cylinder 50 contained in backpack 14 while they are connected to jacket 15 by way of fittings 43 for creating a seal tight hold onto the cylinders 40 and 50 and jacket 15 respectively.

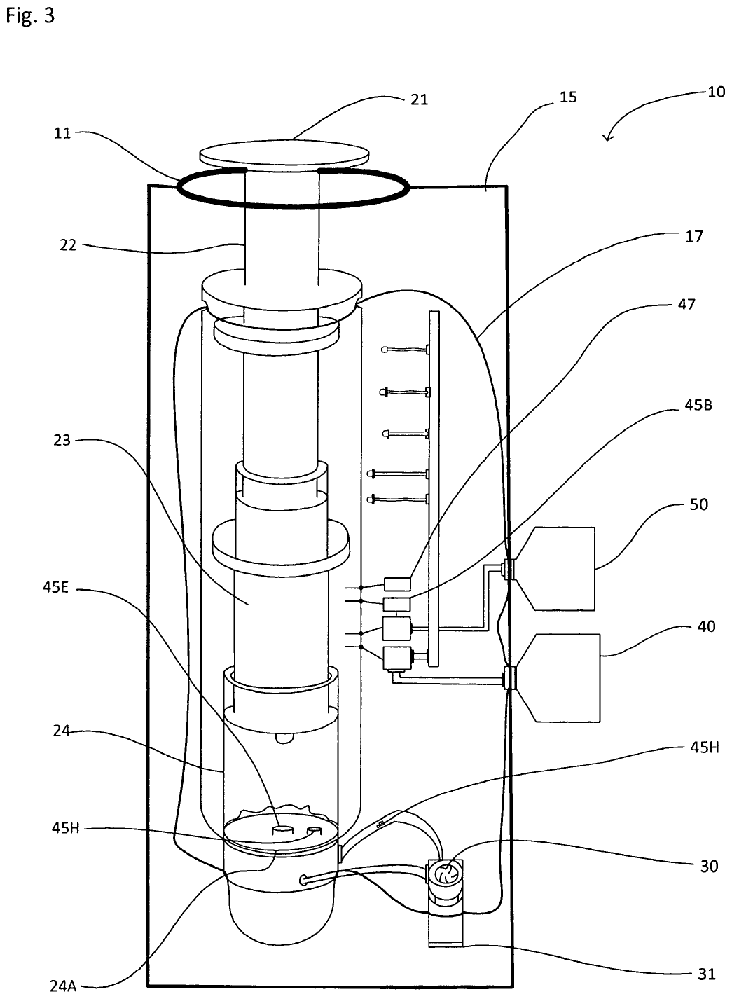

The drawing of FIG. 3 shows the invention housed in jacket 15 and jacket 15 also housed skirt 17. The drawing illustrates, gas cylinder 40 connected by line 42 and fittings 43 to gas flow valve 45A, and line 42 with fittings 43. Tank 40 is showed connected to line 42 with fittings 43 and Valve 45A is showed connected to dispenser chamber 70 by way of line 42 and fittings 43. Dispenser or dispensing chamber 70 is showed connected to hoses 42A for dispensing gas and air therethrough. Hoses 42A is showed comprising, nozzles 72 at its ends for dispensing gas and air in the form of a mist in order to sterilized and sanitized human hands and objects.

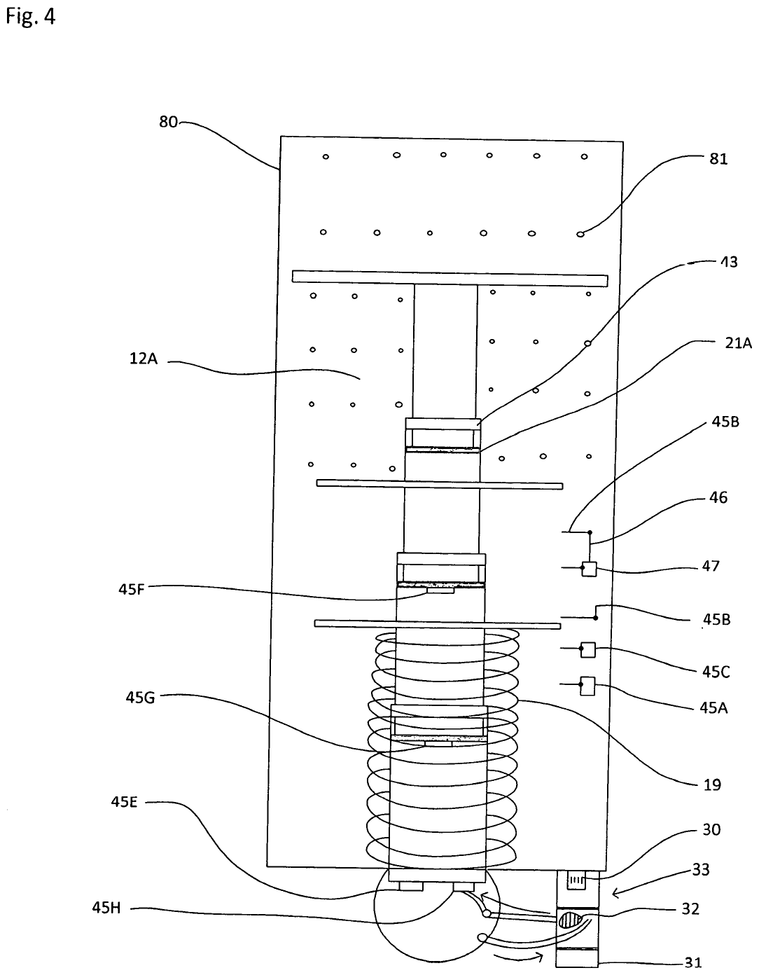

The drawing of FIG. 4 illustrates a section of a single embodiment of the present invention comprising, outer cylinder 80 housing pump 20 therein. The drawing further showed outer cylinder 80 comprising, holes 81 for connecting with nozzles 72, fittings 43 for creating sealed tight connections, downward end 21A of the pump inner member for forcing air downwardly, trigger 45B for triggering the opening and closing of the valves and lock 47, linkage 46 for transferring movements from the triggers to the valves, valve 45C for controlling gas and air flow, valve 45A for controlling gas and air flow, coil spring 19 for urging pump 20 upwardly, fan 30 for pushing outward used air and gas, gear 32 for rotating fan 30, filter 31 for filtering exiting air and gas, one-way reducer valve 45H for reducing return air flow to pump 20, one-way valve 45E for allowing the air from pump to flow in one direction, two-way valves 45G and 45F for allowing air to flow free in one direction and reduce the air flow in the other direction.

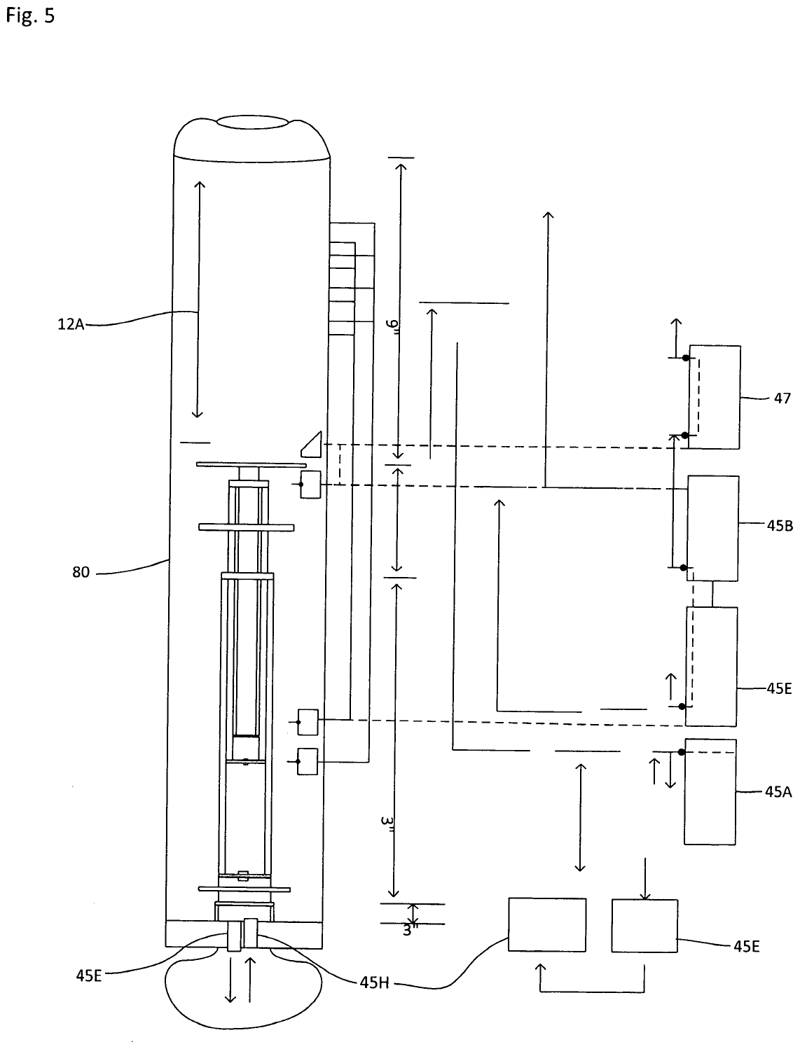

Illustrated in the drawing of FIG. 5, is the flowchart which shows the steps and functions of the invention when in use.

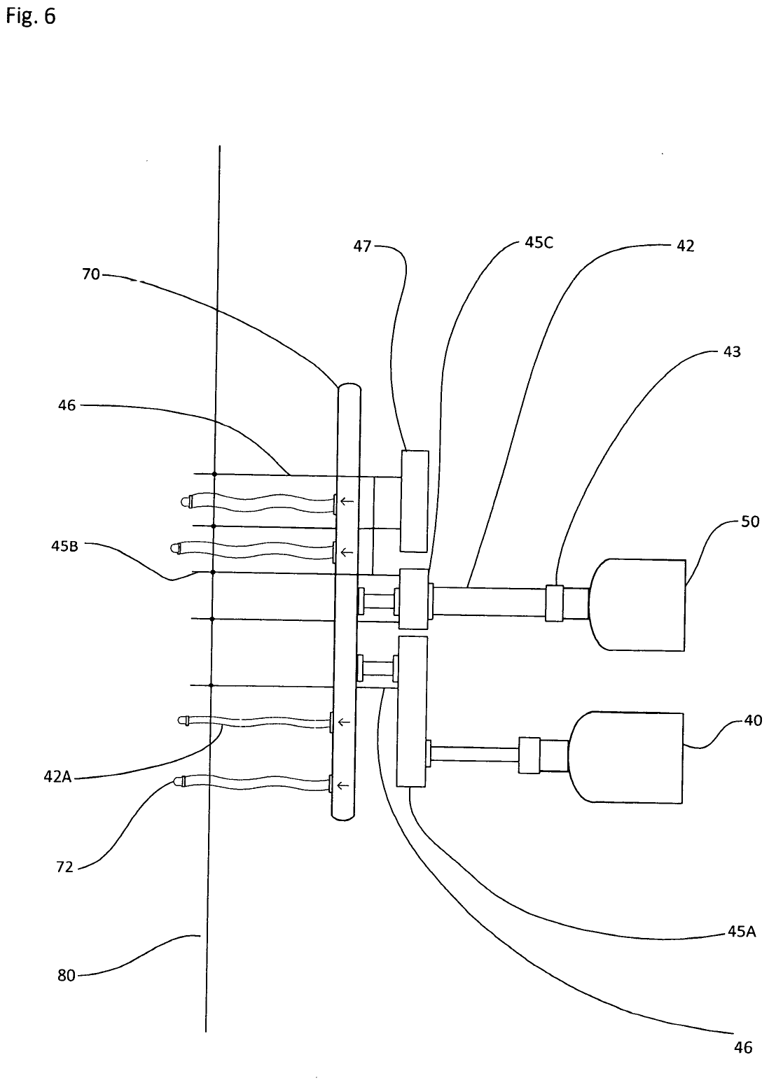

The drawing of FIG. 6, shows a section of the invention connected to cylinders 40 and 50, and comprising: cylinder 40 for sterilizing, cylinder 50 for cleaning, hoses 42 and 42A for providing a pathway for gas and air, fittings 43 for creating connections, valve 45A for controlling sterilizing gas flow, valve 45C for controlling air flow, linkage 46 for linking the members, triggers 45B for triggering movements, lock 47 for keeping pump 20 at a predetermined position for a predetermined amount of time, dispenser 70 for dispensing gas and air and nozzles 72 for enhancing passing gas and air. Hose or Line 42 for carrying compress, gas, air, moisturizing gas and other suitable means for cleaning to dispenser 70 for dispersing.

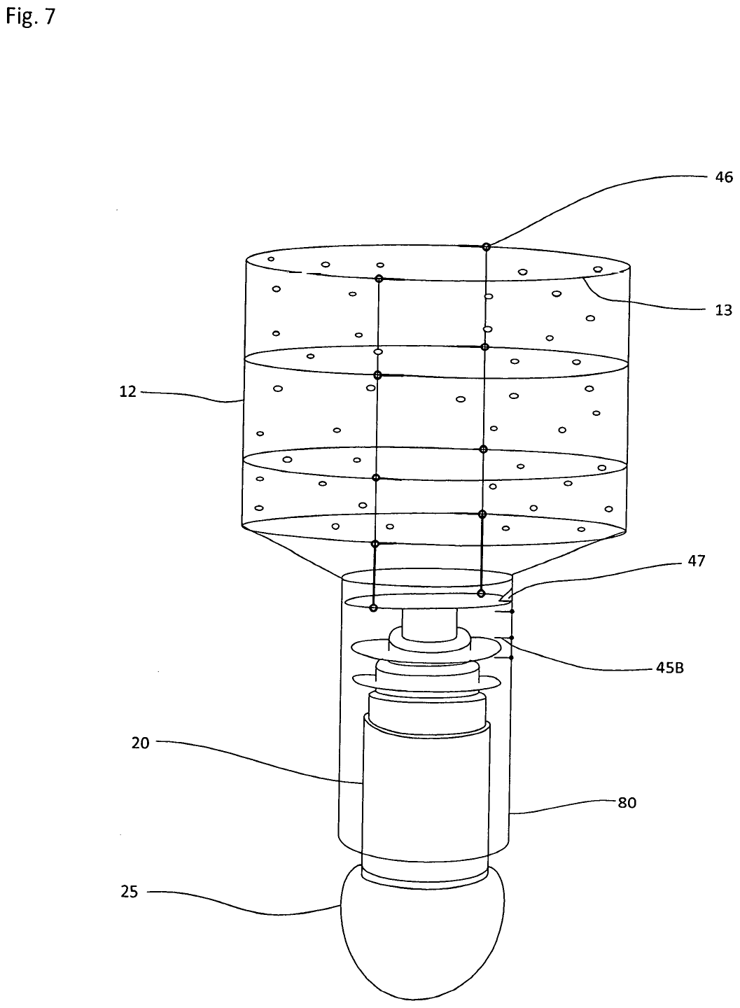

FIG. 7 shows, a transparent and collapsible hand area 12, a transparent outer cylinder 80, a plurality of linkages 46, a plurality of cinctures 13, pump 20 comprising, windbag 25, mechanism for locking 47 and triggers 45B.

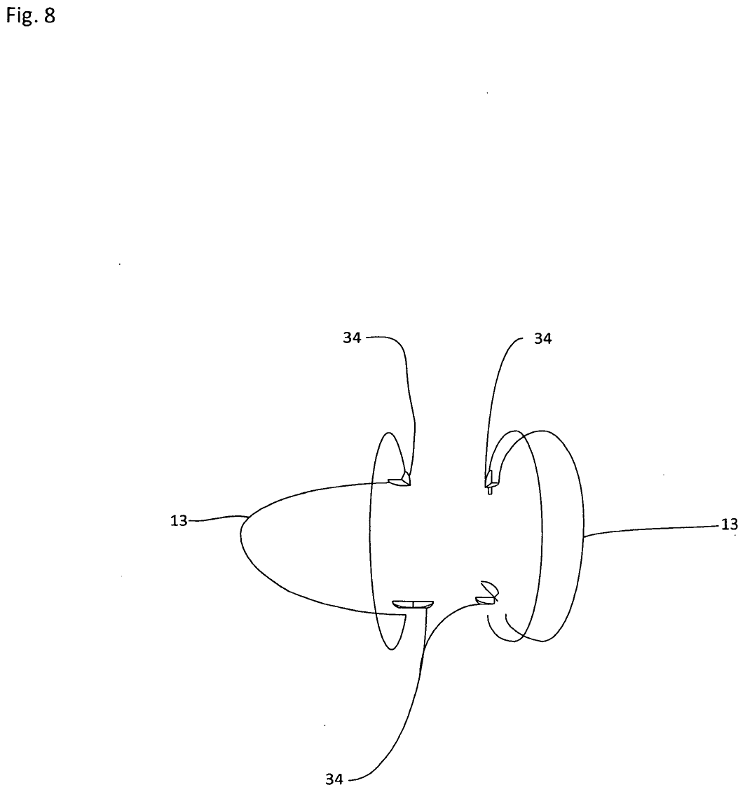

FIG. 8 is a perspective views of, a collapsed or folded cincture 13, and a partially folded cincture 13 comprising, cup hinges 34.

A plurality of small hoses 42A allows for dispensing gas to be transferred from dispenser to nozzles 72 in order to clean hands and objects.

A valve 45A for opening and closing gas flow from cylinder 40 by way of trigger 45B and linkage 46.

A valve 45C for opening and closing cylinder 50 gas flow to dispenser for, dispensing through lines 42A and nozzles 72.

### Detailed Description of Component of the Invention

Backpack 14 houses, the canisters and some hoses 42 and 42A, and is attachable to the jacket of the invention.
Closer 11 (elastic neck) is at the entrance of hand area 12 and is for, closing-inn on anything that is inserted into pocket 16 like a hand or object (not shown in the drawings).
Exhaust fan 30 is adapted to open at predetermined times to exit the used air and gas in cylinder 80, hand area 12 and skirt 17.
Jacket 15 is housed inside vest 18 and further houses most components of the invention except gas cylinders 40 and 50. Dispenser 70 can also be positioned in the backpack outside of, jacket 15.
Skirt 17, seals off pump 20 and outer cylinder 80, for enabling flushing of used gas and air in the system.
Pump 20 is, telescopic in form and comprising, inner member 22 having a closed downward end and push knob 21 at its upward end, second or middle cylindrical member 23 having, outer circumferential first ring gate 22A, a downward end having valve 45F thereon.
Windbag 25 is seal tight attached to the downward end of telescopic member 20 and is for, accommodating a predetermined amount of extra air from the pump and contracting after so that the air can be return to its original place.
Gas cylinder 40 is connected to line 42 by way of fitting 43 and is housed in backpack 14.
Air cylinder 50 is connected to line 42 by way of fitting 43.
Exhaust fan 30 is, connected to cylinder 80, skirt 17 and jacket 15, and is for getting rid of used sterilizing gas and drying air, and also for allowing a predetermined amount of sealed air pressurized by pump 20 to, be sent back to a predetermined location of pump 20 for creating reactive movements
Stationary disc 24A is of pump 20 and comprises valve 45E for, allowing air in one direction only, and reducer valve 45H which allows air in the opposite direction at a reduced rate.
Reducer valve 45H, opens at predetermined times to allow forced air contained In bag 25 to move back to its original position; At the same time the movement of the sealed air will rotate fan 30 thereby pushing out used gas and air from the embodiment. Reducer valve 45H also reduces the flow of returning sealed air back to the pump.
1. Downward movement of trigger 45b of valve 45A will, open valve 45A, and upward movement of trigger 45B will close valve 45A.
2. Upward movement of moisturizing, sanitizing or drying air first trigger 45B will open dry air flow valve 45C so that, dry air can rush through dispenser 70 for dispensing.
3. Upward movement of drying air second trigger 45B will close the flow of drying air from dry air valve 45C.

### CONCLUSION, RAMIFICATION, AND SCOPE

accordingly, the reader will see that the wearable sterilizer apparatus of the invention can be used to sterilize human hands and instruments use to test for infections.

The elements of the present invention conjunct to provide a wearable sterilizer that medical professionals and the public in general can use as a means for sterilizing their hands or objects that may need to be cleaned and or sterilized.

The invention can be used to sterilized children hands, it can be useful in field work by medical professionals and can even be an effective preventive means of avoiding, contracting or transmitting a germ.

The invention is easy to produce, economical and easy to use thereby can be a great addition to preventive measures for our health.

Although the preceding descriptions contain many specificities, these should not be construed as limiting the scope of the invention but as merely providing illustrations of some of the presently preferred embodiments of this invention.

For example, the invention and its components can have many shapes, colors and sizes, other than the ones illustrated in the drawings. The invention can also be made from a plastic material adapted to collapse when nothing is in the pocket.

Thus, the scope of the invention should be determined by the appended claims and their legal equivalents, rather than by the examples given.

The embodiments of the invention in which an exclusive property or privilege is claimed are as follow:

1. A pocket sterilizer that can be carried around, and humans can wear the sterilizer as an over coat and use said pocket sterilizer for sterilizing said humans' hands and other objects which may need to sterilized comprising,
a first means for accommodating the human said hands and said objects which may need to be sterilized,
a second means for sterilizing the hands and said objects,
a third means for crating predetermined movements of predetermined members of the embodiment,
a fourth means for transmission of a predetermined amount of sterilizing gas and a predetermined amount of sterilized air in a compress form, and
a fifth means for transmission of a sealed predetermined amount of said air,
a sixth means for controlling the movements and locations of said members of the embodiment, and said gas and the air, whereby, a person may insert said hands and said objects into said pocket to be sterilized and whereby the pocket sterilizer will sterilized said hands and said objects within a predetermined period of time.

2. The pocket sterilizer as defined in claim 1, in which said first means for accommodating the human said hands and said objects which may need to be sterilized is, the pocket of said sterilizer composed of a durable plastic, having a cylindrical shape and large enough for accommodating the human said hands and said objects that need to be sterilized.

3. The pocket sterilizer as defined in claim 1, in which said second means for sterilizing the hands and said objects is, the sterilizing said gas and said air which will clean and dry said hands and said objects.

4. The pocket sterilizer as defined in claim 1, in which said third means for crating predetermined movements of the gas and said air of the embodiment is, a plurality of canisters which contain the sterilizing said gas and the sterilized said air in a compress form to be used for sterilizing.

5. The pocket sterilizer as defined in claim 4, in which said fourth means for transmission of a predetermined amount of sterilizing gas and a predetermined amount of sterilized air in a compress form is the canisters which contain the sterilizing said gas and the sterilized said air in a compress form to be used for sterilizing,

6. The pocket sterilizer as defined in claim 5, in which said canisters are composed of materials that are resilient and strong enough to hold for dispensing predetermined amount of the sterilizing gas and said air in compress form for cleaning and sterilizing the hands of said humans and said objects that may need to be sterilized.

7. The pocket sterilizer as defined in claim 1, in which said fifth means for transmission of a sealed predetermined amount of said air is a pump.

8. The pocket sterilizer as defined in claim 1, in which said sixth means for controlling the movements and locations of said members of the embodiment are said gas and the air controlling said predetermined movements and locations of said members of the embodiment, and is, a plurality of triggers and linkages for causing the opening and closing of, a plurality of valves which allows and restrict movements of the sterilizing said gas, the sterilized said air and the sealed air.

8. A sterilizing and sanitizing apparatus for the sterilizing and the sanitizing of a plurality of objects and human hands comprising,
a plurality of housing members composed of a plurality of suitable materials, and is adapted to accommodate the hands of said human and the objects,
a plurality of canisters having compress sterilizing said gas and sterilized compress air,
a dispenser for dispensing the compress said gas and said air,
a plurality of valves for opening and closing the flow of the gas and the air,
a plurality of triggers for triggering the opening and the closing of the valves,
A plurality of linkages linking the triggers to said valves, and is for transferring movements of the triggers to said valves,
a plurality of hoses and lines connecting the canisters to the valves, the valves to said dispenser, the dispenser to the lines for dispensing the gas and the air, and for allowing movements of the gas and said air,
a plurality of connectors for connecting the hoses and the lines to the canisters, said dispenser, the valves, the triggers the hoses said lines,
a telescopic member as a pump having an upward end and a downward said end and adapted for forcing said air into,
a windbag containing a predetermined amount of said air is, adapted to expand when the pump is moved downwardly, and contract when the pump moves upwardly, and is attached to the downward end area of said pump,
a plurality of gates which slide to and from predetermined location with the telescopic said member,
a rigid outer cylindrical member large enough to accommodate the human said hands and the objects,
a member for locking and unlocking said at a predetermined location,
whereby said human can place said hands and said objects to be sterilized onto the top outer surface of said pump while pushing downward till said pump comes to a stop, after a predetermined amount of time the telescopic member will push back upward at which time said hands and the objects are sterilized and can be removed from the cylindrical said member and from the top of said pump.

9. A method of sanitizing humans' hands and objects which may need to be sanitized comprising the steps,
a. providing a vest with a pocket which houses a plurality of components for accommodating and sterilizing the hands and said objects,
b. providing backpack for housing a plurality of members for cleaning and sterilizing of said hands and said objects,
c. providing a plurality of canisters for storing compress gas and compress sanitizing air for sterilizing and sanitizing,
d. providing a dispenser chamber for dispensing of a predetermined amount of the gases,
e. providing a plurality of harnesses and straps so that the vest and the backpack can be attachable and wearable by said human,
f. providing a pump comprising, a plurality of gates for triggering opening and closing of the flow of the compress said gas and the air,
g. providing an outer cylindrical member which keeps in place the gates of the pump, and allow for predetermined movements of said pump with said gates,
h. providing a locking and unlocking mechanism which will hold the pump locked at a predetermined location for a predetermined period of time when the pump is pushed downward by a person,
i. providing a windbag containing a predetermined amount of said air and adapted to expand and contract and is, sealed around a predetermined location of said pump, and will cause retraction of the pump when contracted,
j. providing a plurality of valves which will open and close the flow of the gas and the air
k. providing a plurality of mechanisms for triggering the opening and closing of the valves, and for triggering the unlocking of the mechanism for locking and unlocking
l. providing a plurality of hoses for allowing the flowing of the sterilizing said gas and said air,
m. providing a plurality of fittings for connecting the hoses with the canisters, the valves and the dispenser,
whereby, a person using said method of sanitizing the human said hands and said objects, can place said hands and said objects to be sanitized onto the pump and push downwardly until said pump comes to a stop, at which time, said pump will be locked in a downward position for a predetermined period of time by the locking and unlocking said mechanism so that said hands and said objects for sanitizing can be sanitized and, whereby said hands and said objects may be removed after the sanitizing period is finish, and said pump returns to its original starting position.
